SEQUENCE LISTING (5 sequences)

SEQ ID NO: 1 - DNA, 25 bp, Cannabis sativa
  misc_feature (6)..(6): Wherein n at position 6 is a or g
  misc_feature (17)..(17): Wherein n at position 17 is g or c
aaaaanttgtagctcanttgtaact

SEQ ID NO: 2 - DNA, 25 bp, Cannabis sativa
aaaaarttgtagctcasttgtaact

SEQ ID NO: 3 - DNA, 25 bp, Cannabis sativa
aaaaaattgtagctcagttgtaact

SEQ ID NO: 4 - DNA, 20 bp, Artificial Sequence
  Synthetic
taatttcccggctggtgctc

SEQ ID NO: 5 - DNA, 19 bp, Artificial Sequence
  Synthetic
cggccaggctttcgattgc

(19) United States
(12) Patent Application Publication
(10) Pub. No.: US 2021/0363599 A1
(43) Pub. Date: Nov. 25, 2021

(54) SEX IDENTIFICATION OF CANNABIS PLANTS

(71) Applicant: ANANDIA LABORATORIES INC., Vancouver (CA)

(72) Inventors: Jason SAWLER, Centreville (CA); Erin GILCHRIST, Vancouver (CA)

(73) Assignee: ANANDIA LABORATORIES INC., Vancouver, BC (CA)

(21) Appl. No.: 17/252,220

(22) PCT Filed: May 16, 2019
(86) PCT No.: PCT/CA2019/050659

§ 371 Date: Dec. 14, 2020

Related U.S. Application Data
(60) Provisional application No. 62/679,122, filed on Jun. 1, 2018.
(60) Provisional application No. 62/674,852, filed on May 22, 2018.

(51) Int. Cl. C12Q 1/6895; C12Q 1/686

(52) U.S. Cl. CPC C12Q 1/6895; C12Q 2600/13; C12Q 1/686

(57) ABSTRACT

An economical and accurate method for identifying sex of a Cannabis plant involves amplifying an amplicon of genomic nucleic acid molecules from a subject Cannabis plant using polymerase chain reaction (PCR), the amplicon being a genomic region containing the nucleotide sequence as set forth in SEQ ID NO: 1. The sex of the subject Cannabis plant is determined by determining whether SEQ ID NO: 1 is SEQ ID NO: 2 or SEQ ID NO: 3, where SEQ ID NO: 2 is in male plants and SEQ ID NO: 1 is in female plants. Preferably, high resolution melt (HRM) analysis is used to determine the sex of the subject Cannabis plant by comparing the HRM analysis to a control for male or female Cannabis plants. The amplicon can be amplified in both male and female plants with a common primer set.

### CROSS-REFERENCES TO RELATED APPLICATIONS

This application is a National Stage of International Application No. PCT/CA2019/050659, filed May 16, 2019, which claims the benefit of United States Provisional Patent Applications U.S. Ser. No. 62/674,852 filed May 22, 2018 and U.S. Ser. No. 62/679,122 filed Jun. 1, 2018, the entire disclosures of which are herein incorporated by reference.

### STATEMENT REGARDING SEQUENCE LISTING

The sequence listing associated with this application is provided in text format in lieu of a paper copy and is hereby incorporated by reference into the specification. The name of the text file containing the sequence listing is 1018-P11USPNP_Seq_List_FINAL_20210729_ST25.txt. The text file is 47 KB; was created on Jul. 29, 2021, contains no new matter, and is being submitted via EFS Web.

### FIELD

This application relates to methods and primers for identifying the sex of a Cannabis plant.

### BACKGROUND

As Cannabis production becomes increasingly legalized and mainstream worldwide, the large-scale production of uniform Cannabis plants becomes increasingly important from the perspective of product value and quality control. The Cannabis crop is dioecious, with seeds producing separate male and female plants. Male plants are undesirable in crops grown for medical or recreational purposes because they do not typically produce high levels of metabolites. In addition, pollination in the crop leads to seeds in the female plants thereby reducing the value of the harvested product. The ability to readily identify male and female Cannabis plants before flowering is therefore desirable to permit growers to cull the male plants before seeds can be produced, or to separate male and female plants to prevent outcrossing, or to select plants of either sex for breeding purposes. Culling the male plants prevents accidental pollination of female flowers and subsequent production of seeds in female buds. Culling male plants early in the growth cycle also allows growers to free up growth space for the commercially important female plants. In addition, the ability to timely identify male and female plants also assists breeders in selecting plants to use for genetic crosses.

The only published technique, currently, for molecular sex determination in Cannabis (Mandolino 1999) makes use of repeat sequences that are present in both male and female DNA. This technique can result in incorrect identification of males and females unless very specific conditions are used for amplification and gel electrophoresis. In addition, the technique is labor intensive and time consuming.

Other quantitative polymerase chain reaction (qPCR)-based techniques that are currently advertised as being used to identify sex in Cannabis rely on the presence/absence of a product in males/females. The absence of a product produced when primers homologous to the Y chromosome are used, indicates that the plant is female. But the absence of a product could also indicate that the reaction did not work, and even with the use of appropriate controls, there is no way of proving the genotype of female plants due to lack of a visible PCR product.

There remains a need for a more robust and economical method for identifying the sex of Cannabis plants than currently available methods.

### SUMMARY

In one aspect, there is provided a method for identifying sex of a Cannabis plant, the method comprising: amplifying an amplicon of genomic nucleic acid molecules from a subject Cannabis plant using polymerase chain reaction (PCR) to form PCR products, the amplicon comprising the nucleotide sequence as set forth in SEQ ID NO: 1; and, determining the sex of the subject Cannabis plant by determining whether SEQ ID NO: 1 is SEQ ID NO: 2 or SEQ ID NO: 3, the plant being male when SEQ ID NO: 1 is SEQ ID NO: 2 and the plant being female when SEQ ID NO: 1 is SEQ ID NO: 3.

In another aspect, there is provided a method for identifying sex of a Cannabis plant, the method comprising: amplifying an amplicon of genomic nucleic acid molecules from a subject Cannabis plant using polymerase chain reaction (PCR) to form PCR products, the amplicon comprising the nucleotide sequence as set forth in SEQ ID NO: 1; performing high resolution melt (HRM) analysis on the PCR products from the subject Cannabis plant; and, determining the sex of the subject Cannabis plant by comparing the HRM analysis of the PCR products from the subject plant to a control for male or female Cannabis plants.

Sex identification of Cannabis plants using the present method is simple, fast, not labor intensive, reliable and economical allowing identification of both males and females using modern, non-gel-based technology. The method permits identification of the sex of a Cannabis plant prior to flowering, which is particularly useful to growers to allow elimination of male plants where the grower is producing female plants for medical or retail markets. The method is also useful to breeders for developing new germplasm by assisting with timely identification of male and female plants for use in making genetic crosses.

Further features will be described or will become apparent in the course of the following detailed description. It should be understood that each feature described herein may be utilized in any combination with any one or more of the other described features, and that each feature does not necessarily rely on the presence of another feature except where evident to one of skill in the art.

### BRIEF DESCRIPTION OF THE DRAWINGS

For clearer understanding, preferred embodiments will now be described in detail by way of example, with reference to the accompanying drawings, in which:

FIG. 1 depicts analysis of PCR products generated by SEQ ID NO: 4 and SEQ ID NO: 5 as a primer pair using agarose gel electrophoresis at different annealing temperatures.

FIG. 2 depicts a HRM analysis showing differences in fluorescence signal (RFU) vs. shifted temperature (° C.) between male samples (more sinusoidal curves) and female sample (more linear curves).

FIG. 3A depicts a HRM analysis showing differences in fluorescence signal (RFU) vs. shifted temperature (° C.) between male samples (more sinusoidal curves) and female sample (more linear curves) for 20 hemp-type Cannabis plant samples.

FIG. 3B depicts a HRM analysis showing differences in fluorescence signal (RFU) vs. shifted temperature (° C.) between male samples (more sinusoidal curves) and female sample (more linear curves) for 13 drug-type Cannabis plant samples.

### DETAILED DESCRIPTION

Whole genome sequencing analysis of genomic DNA sequences from male and female Cannabis sativa L. plants identified two sex-specific single nucleotide polymorphisms (SNPs) in close proximity to one another in a 119 base pairs (bp) region of the genome. These sequences were verified by Sanger sequencing using primers specific to that region. The region has not been mapped, but the sequence of the surrounding region indicates that they are in a gene that is homologous to the SEUSS-LIKE gene in Arabidopsis, although neither of the SNPs are predicted to have an effect on the putative protein product of the gene. Both of the SNPs are heterozygous in male plants; while both of the SNPs are homozygous in female plants. The nucleotide sequence surrounding the SNPs comprises AAAAANTTGTAGCTCANTTGTAACT (SEQ ID NO: 1), where N at position 6 is A or G and N at position 17 is G or C. In male plants, the nucleotide sequence containing the two SNPs comprises AAAAARTTGTAGCTCASTTGTAACT (SEQ ID NO: 2), where R indicates that the plant is heterozygous for A/G and S indicates that the plant is heterozygous for G/C. In female plants, the nucleotide sequence containing the two SNPs comprises AAAAAATTGTAGCTCAGTTGTAACT (SEQ ID NO: 3). The two sex-specific single nucleotide polymorphisms (SNPs) can be utilized to differentiate between male and female plants.

In the present method, the 119 base pairs (bp) region containing the two sex-specific single nucleotide polymorphisms (SNPs) can be amplified in both male and female plants by polymerase chain reaction (PCR), particularly quantitative polymerase chain reaction (qPCR), with a common primer set followed by determining the sex of the subject Cannabis plant by determining whether SEQ ID NO: 1 is SEQ ID NO: 2 or SEQ ID NO: 3. Any suitable detection technique for single nucleotide polymorphisms (SNPs) may be utilized to detect the SNPs at position 6 and/or position 17 of the SEQ ID NO: 1 to determine whether the subject Cannabis plant is male or female.

In a preferred technique for detecting the SNPs, High Resolution Melt (HRM) analysis (Wittwer 2003) may be conducted in the presence of a common intercalating fluorescent dye to determine whether the plant is male or female. As double stranded DNA denatures during HRM, the loss in fluorescence from the dye can be monitored, the loss in fluorescence over time providing different denaturation curves depending on the whether SEQ ID NO: 2 (male) or SEQ ID NO: 3 (female) is present in the 119 base pairs (bp) region. Comparing the denaturation curve obtained to a control for male and/or female plants permits identifying the sex of the plant. For example, the denaturation curves fall into two clusters, identifying either male or female plants, based on clustering with known male and female positive controls. Because of the sequence differences, and because the male plants carry two heterozygous SNPs, the curves are significantly different, allowing simple and immediate identification of either male or female plant DNA based on this clustering.

Other techniques may be used to detect the SNPs. For example, the TaqMan™ system (Shen 2009) uses an allele-specific primer that recognises the SNP of interest with an allele-specific blocker that covers the wild-type sequence to suppress nonspecific amplification of the nontarget allele. Molecular beacons (Tyagi 1996) uses hairpin-shaped probes that are complementary to a specific DNA sequence and that carry a fluorophore and a quencher, such that when the probe binds to the specific DNA sequence the quencher and the fluorophore are separated and fluorescence emission indicates the presence of the SNP. Allele specific primers or ARMS-PCR type reactions (Newton 1989) may be used in which in a first reaction, a constant primer and a primer complementary to the wildtype template is used and in a second reaction, a constant primer and a primer complementary to the SNP template is used to identify the SNP by the production of a product using a primer specific to the SNP in the second reaction. In the Surveyor/Cel-1 technique (Oleykowski 1998), DNA is amplified using common primers and then digested using an enzyme that recognises single base pair mismatches, the samples run on polyacrylamide or agarose gels to detect the presence of the cleaved product when the SNP is present. In the single-strand conformation polymorphism (SSCP) technique, PCR products are denatured, and separated by gel electrophoresis under nondenaturing conditions, the single-stranded fragment with a SNP having a different conformation from its wild-type counterpart, thus moving differently on the gel. In the restriction fragment length polymorphism (RFLP) technique, PCR products are cleaved using a specific restriction endonuclease that cuts at the SNP site and not the wild-type site (or vice versa), resulting in different fragment lengths when run on a gel. Many other techniques are known in the art for detecting SNPs of interest in a target sequence.

In a preferred embodiment, the common primers are: Forward Primer (EG160f): 5′-TAATTTCCCGGCTGGTGCTC-3′ (SEQ ID NO: 4); and, Reverse Primer (EG162r): 5′-CGGCCAGGCTTTCGATTGC-3′ (SEQ ID NO: 5).

The present method may use any suitable genomic nucleic acid samples from Cannabis, for example samples from seeds, seedlings, tissue cultures or plants of any age. In a preferred embodiment, the samples may comprise Cannabis leaves of seedlings or young plants pressed into a paper matrix, preferably a paper matrix laced with a mixture of chemicals that lyse cells and stabilize nucleic acids on contact for long-term storage at room temperature (e.g. Whatman™ FTA™ cards). The ability to use leaf material pressed into a paper matrix is particularly advantageous because the samples are easily collected, stored at room temperature, and shipped in a format that is not subject to the same restrictions as handling of controlled substances. In addition, expensive and time-consuming DNA extraction procedures are not required when using the present method. In another preferred embodiment, the samples may comprise purified DNA isolated from fresh or dry Cannabis plant material. The purified DNA may be used directly, or may be stored by affixing the purified DNA to filter paper and then used.

### EXAMPLES

Materials and Methods:

Test DNA samples were prepared from Cannabis sativa L. plants of unknown sex as sample discs by pressing leaf tissue from seedlings onto Whatman™ FTA™ cards. DNA controls for male and female genotypes were prepared from Cannabis sativa L. plants of known sex in the same manner as the test samples. Samples were stored at room temperature.

High Resolution Melt Calibration™ Kit and Precision Melt Supermix™ were obtained from Bio-Rad™ and stored at −20° C. Ultrapure water was made as needed and stored at room temperature. EG160f and EG162r primers were synthesised by Eurofins Genomics using sequences we designed. Precision Melt Analysis™ software, Real-Time System™ software and Real-Time Thermocycler System™ were all obtained from Bio-Rad™.

Duplicates of the Whatman™ FTA™ discs for the test samples and controls were cut from the Whatman™ FTA™ test sample and control cards. Duplicate discs containing no plant material (NT negative control) were cut from blank cards. The discs were 1.2 mm in diameter. The discs were washed in PCR tubes or 96-well plates. For each sample, qPCR ingredients were mixed including 5 μL Precision Melt Supermix™, 1 μL Primer Mix (2 μM of each primer), one 1.2 mm disc of the sample or control Whatman™ FTA™ card and 4 μL ultrapure water, ensuring that the disc was completely submerged. The tubes or plates were sealed and placed in the thermocycler for qPCR amplification. Amplification was performed as follows: 95° C. for 2 min for initial denaturation; then 40 cycles of 95° C. for 10 sec for denaturation; 59° C. for 30 sec for annealing and extension. While annealing was performed at 59° C., the annealing step may be suitably performed at a temperature in a range of 55° C. to 65° C. Then samples were heated to 95° C. for 30 sec for denaturation; then kept at −60° C. for 1 min for the final extension step.

After amplification, the qPCR products were heated from 70° C. to 80° C. at 0.2° C. increments each 10 sec with fluorescence readings being taken. Loss of fluorescence is recorded as the double stranded PCR products are denatured. The rate of fluorescence loss is used to cluster the denaturation curves into two groups that can be identified as either male or female by comparison with the positive controls. Analysis of the data collected was performed using the Precision Melt Analysis™ software.

The procedure was used to identify the sex of 25 hemp-type cannabis plants and 17 drug-type cannabis plants, whose sex was already known, to confirm accuracy and to make sure that the procedure was accurate for both hemp and drug-type cannabis plants. 123 samples were tested using both the Mandolino technique and the present qPCR-HRM technique to confirm that the present procedure is as accurate as the previously published technique. A total of 164 cannabis samples were tested altogether (81 samples in triplicate and 83 samples in duplicate).

### Results:

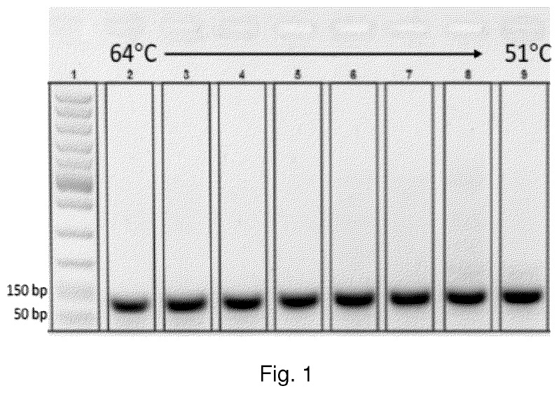

The primer pair (EG160f and EG162r) was tested by regular PCR and subsequent agarose gel electrophoresis to confirm successful amplification using annealing temperatures between 51° C. and 64° C. As seen in FIG. 1, single amplicons of the expected fragment size of 119 bp were observed in all samples, indicating that the correct DNA region was amplified. Subsequent Sanger sequencing of the PCR products showed that only the DNA region containing the sex-specific SNPs was amplified. Using 20 ng of purified DNA as template resulted in sufficient amplification with Cₜ values between 20 and 30 cycles. Applying a melting curve analysis after the qPCR run resulted in a single melting peak confirming that a single PCR product was amplified and no primer dimers were formed.

The optimal temperature range for melting of the heteroduplex for HRM analysis was found to be from 70° C. to 80° C. (in 0.2° C. increments). A temperature range of 65° C. to 95° C. did not provide any additional information, as all molecules had melted by the time the temperature reached 80° C.

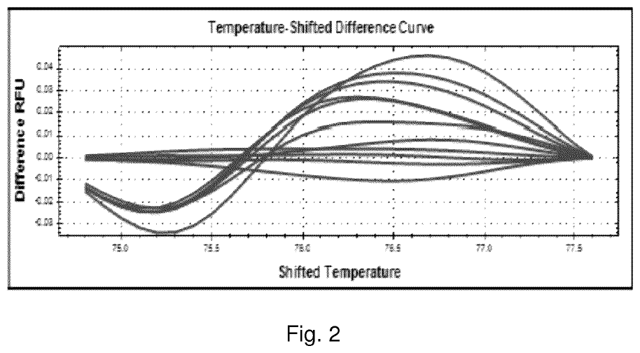

Using FTA disks instead of purified DNA as template, resulted in background noise (unusual amplification curve shapes), likely caused by reflections of the FTA paper disk. Using purified DNA did not result in background noise. Using smaller FTA disks (1.2 mm) as templates for the HRM analysis resulted in slightly less background noise compared to bigger FTA disks (2 mm). Despite the background noise caused by the FTA disks during amplification, HRM analysis of female and male samples showed a consistent difference in melting behavior. By plotting the difference of fluorescence signal between the samples at each temperature point, female and male samples show a different curve shape (FIG. 2). While the male samples produced more sinusoidal curves and the female samples produced more linear curves, the shape of the curves depend on which is the control cluster, and the shapes may be reversed depending on the method being used. Based on differences in fluorescence signals at certain melting temperatures the female and male samples were correctly clustered into two groups with high confidence, confirming that FTA disks can be used as templates for the HRM analysis. Samples with unusual amplification curves and NT controls are always excluded from the analysis during our quality control processing.

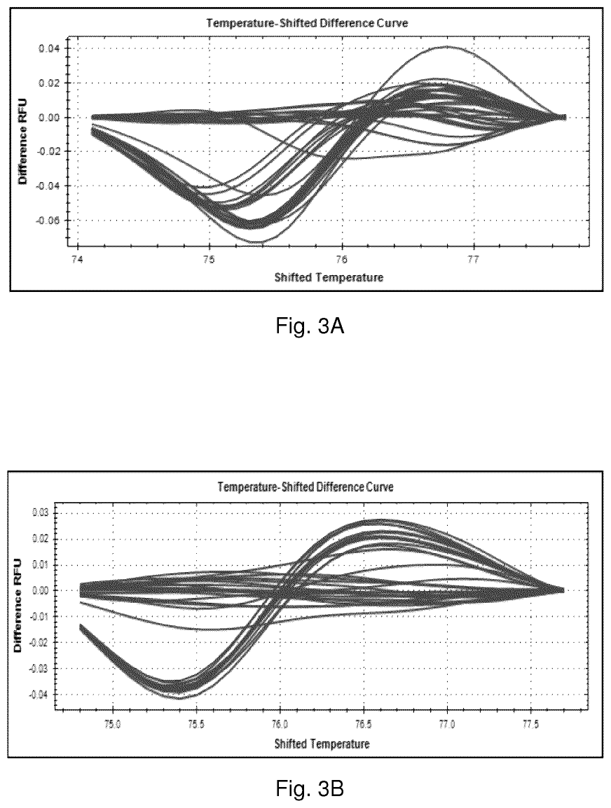

As seen in FIG. 3A and FIG. 3B, the qPCR-HRM method can be used for sex identification in both hemp-type and drug-type Cannabis plants. In both types of Cannabis plants, the same distinct differences are seen in the HRM curves produced in the HRM analysis.

Male and female flowers are easily differentiated visually, which was used to confirm the accuracy of sex identification using HRM analysis. A total of 42 plants were phenotypically identified as either male or female and all 42 were identified correctly by HRM analysis. In addition, a total of 123 samples were tested using both the Mandolino technique and the present qPCR-HRM technique and the same result was seen in all 123 samples, proving that the present technique is as reliable as the previously published technique.

Further, running samples in triplicate or duplicate demonstrated that two replicates per sample are sufficient to verify the sex of each sample. Furthermore, excluding samples from the HRM analysis showing low quality amplification during qPCR, including negative controls, running samples in duplicates and using a Percent Confidence threshold >96% for clustering into female or male categories proved to be sufficient to exclude false results.

Further optimization of the validation dispenses with the need to run samples in duplicate.

### REFERENCES

The contents of the entirety of each of which are incorporated by this reference.
Bio-Rad Technical Paper. What is High Resolution Melting (HRM)? May 8, 2018. www.bio-rad.comien-ca/applications-technologies/what-high-resolution-melting-hrm?ID=LUSOIH97Q.
Garritano S, al, (2009). Determining the effectiveness of high resolution melting analysis for SNP genotyping and mutation scanning at the TP53 locus. BMC Genet. 10, 5.
Han Y, Khu D-M, Monteros M J. (2012). High-resolution melting analysis for SNP genotyping and mapping in tetraploid alfalfa (Medicago sativa L.). Molecular Breeding. 29(2), 489-501.
Li F, Mu B, Huang Y, Meng Z. (2012) Application of High-Resolution DNA Melting for Genotyping in Lepidopteran Non-Model Species: Ostrinia furnacalis (Crambidae), PLoS ONE 7(1): e29664.
Mandolino G, Carboni A, Forapani S. Faeti V, Rana P. (1999) identification of DNA markers linked to the male sex in dioecious hemp (Cannabis sativa L). TAG Theoretical and Applied Genetics, 98(1), 88-92.
Newton C R, Graham A, Heptinstall L E, Powell S J, Summers C, Kalsheker N, Smith J C, Markham A F. (1989) Analysis of any point mutation in DNA, The amplification refractory mutation system (ARMS). Nucleic Acids Research. 17(7), 2503-2516.
Oleykowski C A, Mullins C R B, Godwin A K, Yeung A T. (1998) Mutation detection using a novel plant endonuclease.” (PDF). Nucleic Acids Research. 26(20), 4597-4602.
Shen G Q, Abdullah K G, Wang Q K. (2009) The TaqMan™ Method for SNP Genotyping. In: Komar A. (eds) Single Nucleotide Polymorphisms. Methods in Molecular Biology (Methods and Protocols), vol 578. Humana Press, Totowa, N.J.
Tyagi S, Kramer F R. (1996) Molecular Beacons: Probes that Fluoresce upon Hybridization. Nature Biotechnology. 14, 303-308.
Wittwer C T, Reed G H, Gundry C N, Vandersteen J G, Pryor R J (2003). High-resolution genotyping by amplicon melting analysis using LCGreen. Clin. Chem. 49(6 Pt 1), 853-60.

The novel features will become apparent to those of skill in the art upon examination of the description. It should be understood, however, that the scope of the claims should not be limited by the embodiments but should be given the broadest interpretation consistent with the wording of the claims and the specification as a whole.

1. A method for identifying sex of a Cannabis plant, the method comprising:
amplifying an amplicon of genomic nucleic acid molecules from a subject Cannabis plant using polymerase chain reaction (PCR) to form PCR products, the amplicon comprising the nucleotide sequence as set forth in SEQ ID NO: 1; and,
determining the sex of the subject Cannabis plant by determining whether SEQ ID NO: 1 is SEQ ID NO: 2 or SEQ ID NO: 3, the plant being male when SEQ ID NO: 1 is SEQ ID NO: 2 and the plant being female when SEQ ID NO: 1 is SEQ ID NO: 3.

2. The method according to claim 1, wherein determining whether SEQ ID NO: 1 is SEQ ID NO: 2 or SEQ ID NO: 3 is accomplished by high resolution melt (HRM) analysis on the PCR products.

3. The method according to claim 1, wherein determining whether SEQ ID NO: 1 is SEQ ID NO: 2 or SEQ ID NO: 3 is accomplished by:
using an allele-specific primer that recognizes a single nucleotide polymorphism of interest in the PCR products with an allele-specific blocker that covers SEQ ID NO: 1 to suppress nonspecific amplification of a nontarget allele; using molecular beacons on the PCR products;
using ARMS-PCR type reactions on the PCR products;
using Surveyor/Cel-1 technique on the PCR products;
using single-strand conformation polymorphism (SSCP) technique on the PCR products; or,
using restriction fragment length polymorphism (RFLP) technique on the PCR products.

4. A method for identifying sex of a Cannabis plant, the method comprising:
amplifying an amplicon of genomic nucleic acid molecules from a subject Cannabis plant using polymerase chain reaction (PCR) to form PCR products, the amplicon comprising the nucleotide sequence as set forth in SEQ ID NO: 1;
performing high resolution melt (HRM) analysis on the PCR products from the subject Cannabis plant; and,
determining the sex of the subject Cannabis plant by comparing the HRM analysis of the PCR products from the subject plant to a control for male or female Cannabis plants.

5. The method according to claim 4, wherein the HRM analysis is performed in the presence of an intercalating fluorescent dye with a denaturing temperature increasing from 70° C. to 80° C. in 0.2° C. increments.

6. The method according to claim 5, wherein loss in fluorescence of the fluorescent dye over time provides different denaturation curves depending on the whether the plant is male or female, and the sex of the plant is determined by comparing a denaturation curve obtained from the plant to a control for male and/or female plants.

7. The method according to claim 1, wherein the amplicon comprises the sequence as set forth in SEQ ID NO: 2 if the subject Cannabis plant is male.

8. The method according to claim 1, wherein the amplicon comprises the sequence as set forth in SEQ ID NO: 3 if the subject Cannabis plant is female.

9. The method according to claim 1, wherein the amplicon is amplified with a forward primer having the nucleotide sequence as set forth in SEQ ID NO: 4 and a reverse primer having the nucleotide sequence as set forth in SEQ ID NO: 5.

10. The method according to claim 1, wherein the amplicon is a genomic 119 base pair region containing the sequence as set forth in SEQ ID NO: 1.

11. The method according to claim 1, wherein the polymerase chain reaction (PCR) is quantitative polymerase chain reaction (qPCR).

12. The method according to claim 1, wherein the amplifying comprises an annealing step performed at a temperature in a range of 55° C. to 65° C.

13. The method according to claim 1, wherein the subject Cannabis plant is Cannabis sativa L.

14. The method according to claim 1, wherein the amplicon of genomic nucleic acid molecules from the subject Cannabis plant is obtained from a seed, seedling, tissue culture or plant of any age.

15. The method according to claim 1, wherein the subject Cannabis plant is a seedling or young plant and the genomic nucleic acid molecules are provided in samples of leaves from the seedling or young plant pressed into a paper matrix.

16. The method according to claim 15, wherein the paper matrix comprises a mixture of chemicals that lyse cells and stabilize nucleic acids on contact for storage at room temperature.

17. A primer pair comprising SEQ ID NO: 4 and SEQ ID NO: 5.
